(19) United States
(12) Patent Application Publication
(10) Pub. No.: US 2008/0249639 A1
(43) Pub. Date: Oct. 9, 2008

(54) Medical Artificial Nerve Graft Containing Silk Fibroin and Its Preparation Method

(76) Inventors: Xiaosong Gu, Jiangsu (CN); Yumin Yang, Jiangsu (CN); Fei Ding, Jiangsu (CN); Peiyun Zhang, Jiangsu (CN); Yan Liu, Jiangsu (CN); Mei Liu, Jiangsu (CN); Xuemei Chen, Jiangsu (CN); Xiaodong Wang, Jiangsu (CN)

Correspondence Address: THOMAS, KAYDEN, HORSTEMEYER & RISLEY, LLP, 600 GALLERIA PARKWAY, S.E., STE 1500, ATLANTA, GA, 30339-5994, US

(21) Appl. No.: 12/088,167

(22) PCT Filed: Dec. 31, 2005
(86) PCT No.: PCT/CN2005/002423

§ 371 Date: Mar. 26, 2008

(30) Foreign Application Priority Data
Sep. 28, 2005 (CN) 200510094683.2

(51) Int. Cl. A61F 2/02; A61B 17/08

(52) U.S. Cl. 623/23.76; 623/11.11; 606/151

(57) ABSTRACT

A medical artificial nerve graft containing silk fibroin used in bridging of nerve damage and its preparation method. The medical artificial nerve graft comprised of vessel or comprised of vessel and fibre scaffold, wherein at least one of the vessel and the fibre scaffold contains component of silk fibroin, and the wall of the vessel has three-dimensional structure provided with numerous microaptures. The preparation method includes the following steps: silk is used to prepare silk fibre; the silk fibroin fibre is used to prepare the vessel or the fibre scaffold containing silk fibroin; and combining the fibre scaffold with the vessel i.e. inlaying the fibre scaffold into the vessel and so on.

### FIELD OF THE INVENTION

The present invention relates to a medical artificial nerve graft used in bridging of nerve damage and its preparation method.

### BACKGROUND OF THE INVENTION

Clinically peripheral nerve coloboma resulting from trauma (or surgery) is seen more often. But when long-distant nerve coloboma cannot be make up by the method of end-to-end sutured, it is necessary to use a graft in bridging. Although seeking and studying a more suitable graft has been more than one hundred years, people still failed to find an ideal nerve graft which can be mostly recognized and widely used in clinic, except that auto-nerve has become the first choice of graft in bridging of nerve coloboma. The auto-nerve graft fails in extensive clinical application due to limited nerve graft source, difficult match of tissue structure and size, long-term denervation in the graft region.

The emergency and developing of tissue engineering provides a new path for constructing auto-nerve graft. In recent over 20 years, people have tried to use some biodegradable materials in the peripheral nerve tissue engineering, usually some high polymers, including their copolymers and derivates of more than 10 kinds, such as polyglycolic acid (PGA), polylactic acid (PLA), chitosan, gelatin, silica gel, etc. (e.g., China Patent CN1126515C, etc.). But they have different disadvantages in practical application.

### SUMMARY OF THE INVENTION

### Technology:

The objective of the present invention is to provide a medical artificial nerve graft containing silk fibroin with good effect, good biocompatibility with human body, and excellent biodegradation properties and its preparation method.

Technic proposal:

The technic proposal of the present invention is as follows.

A medical artificial nerve graft containing silk fibroin comprises vessel or comprises vessel and fiber scaffolds, wherein at least one of the vessel and the fiber scaffold contains the component of silk fibroin.

The vessel contains silk fibroin component. The vessel also contains chitosan. The fiber scaffold is the silk fibroin fiber.

A preparation method of a medical artificial nerve graft containing silk fibroin, wherein, includes the following steps:

(1) Preparing the silk into the silk fibroin fiber;

(2) Preparing the silk fibroin fiber into the vessel containing silk fibroin.

Chitosan is also added in step (2), and the prepared vessel containing silk fibroin may also contain chitosan.

The prepared vessel also combines with the fiber scaffold, i.e., inlaying the fiber scaffold into the vessel containing silk fibroin. The fiber scaffold is the silk fibroin fiber.

A preparation method of a medical artificial nerve graft containing silk fibroin, wherein, includes the following steps:

(1) Silk is used to prepare silk fibroin fiber to obtain silk fibroin fiber scaffold;

(2) Inlaying the silk fibroin fiber scaffold into the vessel containing PGA, PLA, chitosan or chitin.

### Advantages:

Natural silk fibroin is a traditional feeding product in China. It is also the main species in fibroin fiber. The fibroin accounts for 70 percent to 80 percent of the silk weight. It is the essential material that people use. The fibroin comprises 18 amino acids. Our experimental studies show it has good bioaffinity, non-toxicity, non-pollution, biogradation. The prepared artificial nerve graft has better effect. According to the literature retrieval, no artificial nerve graft prepared from silk fibroin has been found to treat nerve damage at home and abroad. The material of the product in the present invention is a high purity silk fibroin. It is natural degradable material and has good biocompatibility with human body. The prepared product contains no exogenous substance with toxic or side effect due to preparation technologies. The wall of the vessel has rich dimensional structure provided with numerous micropore. It provides necessary path for nutrient transfer needed in the neurocyte growth process, with good effect obtained. It provides essential induction and necessary growth space for neurocyte growth.

We co-cultured the prepared product with nerve tissue cells in vitro. The product was found to have good histocompatibility after morphological observation, determination of acid metabolic activity, determination of nerve growth factor expression. The product was in vivo used to repair a 3-cm coloboma in rat sciatic nerve. It was found to be advantageous to the nerve regeneration, and the recovery of the damaged nerve function. Meanwhile, the product had good biocompatibility.

### DETAILED DESCRIPTION OF THE EMBODIMENTS

The present invention is further illustrated with detailed embodiments as follows:

### Embodiment 1

(1) The preparation of silk fibroin fiber (prepared according to routine methods or purchased from finished product of silk fibroin fiber) or preparation as the following methods: Use the temperature of 50-100 degree Celsuis to heat treatment the silk in alkalescent solution (0.1-10 percent sodium carbonate or 0.1-10 percent potassium carbonate). Then wash the treated fiber in distilled water and obtain the silk fibroin fiber, part of the fiber is used in step (2), while the other is used in step (3) as the fiber scaffold.

(2) The preparation of a silk fibroin vessel: Dissolve part of the prepared silk fibroin fiber in calcium chloride-alcohol-water ternary solutions (the molar ratio of calcium chloride-alcohol-water is 1:2:8) at 25-80 degree Celsius (such as 25 degree Celsius, 50 degree Celsius, and 80 degree Celsuis) for 0.5-6 h (such as 0.5 h, 3 h, and 6 h). Fill the dissolved mixture solution in the cellulose membrane bags and dialyze it with distilled water. Inject the dialyzed fibroin solution into the mould, and conduct modeling process after drying. Then put the preliminarily mould fibroin vessel in the solution of methanol and sodium hydrate(1 mol/L) with 50:50(V:V) for a certain time (5 min-2 h), then wash it with sodium hydrate (1 mol/L), 50 mmol/L phosphate buffer solution, distilled water in order, and obtain the silk fibroin fiber vessel.

(3) Combination: Inlay the obtained silk fibroin fiber vessel into the vessel containing silk fibroin, and combine the medical artificial nerve graft containing silk fibroin.

### Embodiment 2

A preparation method of a medical artificial nerve graft containing silk fibroin, wherein, includes the following steps:

(1) The preparation of a silk fibroin vessel: Obtain the silk fibroin vessel in the same method as embodiment 1.

(2) Directly using the vessel containing silk fibroin in nerve regeneration, or using it in nerve regeneration after filling other substance into it. Or inlaying the prepared silk fibroin vessel into the fiber scaffold which made by other materials (such as PGA, PLA, chitosan, chitin) then using it in nerve regeneration. See contents of China Patent CN1126515C for the fiber scaffold preparation methods by PGA, PLA, chitosan. The method for the fiber scaffold prepared from chitin is as follows: Dissolving chitosan in the weak acidic solution, such as acetic acid, or phosphoric acid, citric acid, lactic acid at the concentration of 2-15 percent (such as 2 percent, 8 percent, and 15 percent). Then processing the solution into the chitosan fiber using wet spinning to obtain chitosan fiber. Putting the obtained chitosan fiber into the alcoholic solution containing acetic anhydride to have acetylation reaction at 0-60 degree Celsius (such as 0 degree Celsius, 30 degree Celsius, and 60 degree Celsius) for 0.5-6 h (such as 0.5 h, 2 h, 4 h, and 6 h). In the alcoholic solution containing acetic anhydride, the concentration of acetic anhydride is 1-50 percent (such as 1 percent, 20 percent, 40 percent, and 50 percent). The alcoholic solution is methanol (or alcohol). Washing it with water after acetylation reaction, and putting it in 1 mol/L sodium hydrate or potassium hydrate for 24 h. Washing it with water and obtain the chitin fiber (i.e., fiber scaffold) after drying.

### Embodiment 3

A preparation method of a medical artificial nerve graft containing silk fibroin, wherein, includes the following steps:

(1) The preparation of a silk fibroin vessel: Dissolving the silk fibroin fiber obtained in the same method as embodiment 1 in calcium chloride-alcohol-water ternary solutions (the molar ratio of calcium chloride-alcohol-water is 1:2:8) at 25-80 degree Celsius (such as 25 degree Celsius, 50 degree Celsius, and 80 degree Celsius) for 0.5-6 h (such as 0.5 h, 2 h, and 6 h). Filling the dissolved mixture solutions in the cellulose membrane bags, dialyzing it with the distilled water, and obtaining the silk fibroin solution.

Dissolving chitosan in the weak acidic solution such as acetic acid, or phosphoric acid, citric acid, lactic acid at the concentration of 2-15 percent (such as 2 percent, 8 percent, and 15 percent). Then mixing up the chitosan solutions in the silk fibroin solution at certain ratio of weight (such as 1:2, 1:1, and 2:1). Then injecting the aforementioned mixture solution in a mould, and conducting modeling process after cryodesiccation. Then putting the preliminarily mould fibroin vessel in the solution of methanol and sodium hydrated mol/L) with 50:50(V:V) for a certain time (5 min-2 h), then washing it with sodium hydrate (1 mol/L), 50 mmol/L phosphate buffer solution, and distilled water in order, and obtaining the complex vessel containing silk fibroin.

(2) Directly using the vessel containing silk fibroin in nerve regeneration, or using it in nerve regeneration after filling other substance into it. Or inlaying the prepared silk fibroin vessel into the fiber scaffold which made by other materials (such as PGA, PLA, chitosan, chitin) then using it in nerve regeneration.

### Embodiment 4

A preparation method of a medical artificial nerve graft containing silk fibroin, wherein, includes the following steps:

(1) Obtaining the silk fibroin fiber (i.e., fiber scaffold) in the same method as embodiment 1.

(2) Inlaying the prepared fiber scaffold into the silk fibroin vessel which made by other materials (such as PGA, PLA, chitosan, chitin) then using it in nerve regeneration. See the contents of China Patent CN1126515C for the vessel preparation methods by PGA, PLA, chitosan. The method for the vessel prepared from chitin is as follows: Dissolving chitosan in the weak acidic solution, such as acetic acid, or phosphoric acid, citric acid, lactic acid at the concentration of 2-15 percent(such as 2 percent, 8 percent, and 15 percent). Then letting the solution pass through the tubiform mould, and shaping in alkaline coagulation solution such as solution containing 8-20 percent sodium hydrate (or 8-20 percent potassium hydrate, or 8-20 percent sodium carbonate, and it may also contain 5-30 percent alcohol). Washing the vessel with water after moulding, and obtaining the chitosan vessel. Then putting the obtained chitosan vessel in the alcoholic solution containing acetic anhydride to have acetylation reaction at 0-60 degree Celsius (such as 0 degree Celsius, 30 degree Celsius and 60 degree Celsius) for 0.5-6 h (such as 0.5 h, 2 h, 4 h, and 6 h) (The reaction time and temperature may affect the acetylation degree of chitin. The longer reaction time and the higher reaction temperature get chitin with the higher acetylation degree. Conversely, get the chitin with lower acetylation degree. The chitin with different acetylation degree can be got under the necessity. The concentration of acetic anhydride in the alcoholic solution is 1-50 percent (such as 1 percent, 20 percent, 40 percent, and 50 percent). The alcoholic solution is methanol (or alcohol). Washing it with water after acetylation reaction, and putting it in 1 mol/L sodium hydrate or potassium hydrate for 24 h. Washing it with water and obtaining the chitin fiber scaffold after drying.

What is claimed is:

1. A medical artificial nerve graft containing silk fibroin comprises vessel or comprises vessel and fiber scaffolds, wherein at least one of the vessel and the fiber scaffold contains component of silk fibroin.

2. A medical artificial nerve graft containing silk fibroin as claimed in claim 1, wherein, the vessel contains silk fibroin component.

3. A medical artificial nerve graft containing silk fibroin as claimed in claim 2, wherein, the vessel contains chitosan.

4. A medical artificial nerve graft containing silk fibroin as claimed in claim 1, wherein, the fiber scaffold is the silk fibroin fiber.

5. A preparation method of a medical artificial nerve graft containing silk fibroin as claimed in claim 1, wherein, includes the following steps:
(1) Preparing the silk into the silk fibroin fiber;
(2) Preparing the silk fibroin fiber into the vessel containing silk fibroin.

6. A preparation method of a medical artificial nerve graft containing silk fibroin as claimed in claim 5, wherein, chitosan is also added in step (2), and the prepared vessel containing silk fibroin may also contain chitosan.

7. A preparation method of a medical artificial nerve graft containing silk fibroin as claimed in claim 5, wherein, the prepared vessel also combines with the fiber scaffold.

8. A preparation method of a medical artificial nerve graft containing silk fibroin as claimed in claim 7, wherein, the fiber scaffold is the silk fibroin fiber.

9. A preparation method of a medical artificial nerve graft containing silk fibroin as claimed in claim 1, wherein, includes the following steps:
(1) Silk is used to prepare silk fibroin fiber to obtain silk fibroin fiber scaffold;
(2) Inlaying the silk fibroin fiber scaffold into the vessels containing PGA, PLA, chitosan or chitin.
